(19) United States
(12) Patent Application Publication
(10) Pub. No.: US 2026/0332272 A1
(43) Pub. Date: Oct. 22, 2026

(54) SIGNAL PROCESSING DEVICE, SIGNAL PROCESSING METHOD, PROGRAM, AND LEARNING DEVICE

(71) Applicant: SONY GROUP CORPORATION, Tokyo (JP)

(72) Inventors: Takanori ISHIKAWA, Saitama (JP); Ryotaro MATSUKAWA, Tokyo (JP); Yasuhide HYODO, Tokyo (JP); Kiyoshi YOSHIKAWA, Saitama (JP)

(73) Assignee: SONY GROUP CORPORATION, Tokyo (JP)

(21) Appl. No.: 19/109,772

(22) PCT Filed: Sep. 6, 2023
(86) PCT No.: PCT/JP2023/032461

§ 371 Date: Mar. 7, 2025

(30) Foreign Application Priority Data
Sep. 21, 2022 (JP) 2022-149751
Jun. 8, 2023 (JP) 2023-094921

(51) Int. Cl. H04B 13/00; H04B 10/80; H04B 15/00; H04B 17/309

(52) U.S. Cl. CPC H04B 13/005; H04B 10/80; H04B 15/005; H04B 17/346

(57) ABSTRACT

A heart rate can be estimated with high accuracy. A signal processing device estimates a first signal quality of an input biometric signal; estimates a second signal quality of an output signal from a noise reduction processor; estimates a heart rate based on the output signal from the noise reduction processor, and estimates a reliability of the estimated heart rate based on a result of the estimated first signal quality and a result of the estimated second signal quality; and corrects the estimated heart rate based on the reliability of the estimated heart rate. The present technology can be applied to a wearable heart rate meter.

### CROSS REFERENCE TO RELATED APPLICATIONS

This application claims the benefit of Japanese Priority Patent Application JP 2022-149751 filed on Sep. 21, 2022, and the benefit of Japanese Priority Patent Application JP 2023-094921 filed on Jun. 8, 2023, the entire contents of which are incorporated herein by reference.

### TECHNICAL FIELD

The present technology relates to a signal processing device, a signal processing method, a program, and a learning device and more particularly, to a signal processing device, a signal processing method, a program, and a learning device capable of estimating a heart rate with high accuracy.

### BACKGROUND ART

Wearable devices equipped with a heart rate measurement function are currently widely used in various applications such as healthcare and heart rate training.

A heart rate measurement function mounted on a wearable device may use photoplethysmography (hereinafter, referred to as a PPG method). The PPG method is a method in which light rays emitted from a light emitting unit (for example, a light emitting diode (LED)) are absorbed, scattered, and reflected by blood and subdermal tissues existing several mm below the skin, and the amount of reflected light is measured by a light receiving unit (for example, a photo-detector) to measure a signal (hereinafter referred to as pulse wave signal) indicating a change in blood flow of a capillary blood vessel distributed below the skin.

Heart rate variability is one known physiological indicator reflecting autonomic nerve activity. In the PPG method, heart rate variability is analyzed by time series data during a time interval of a peak position of a pulse wave signal.

In the case of using the PPG method, the pulse wave signal may be measured with relatively high accuracy in a resting state where the measurement site hardly moves, but when the measurement site moves, noise (hereinafter, referred to as body motion noise) occurs in the pulse wave signal. The body motion noise in the wristband-type PPG type heart rate sensor (hereinafter, referred to as a PPG sensor) is a change in the contact state between the PPG sensor and the measurement site, the following are the four main factors that contribute to body motion noise.
(1) Mixing of unnecessary reflected light on the skin surface;
(2) Mixing of external light transmitted under the skin;
(3) Pseudo signal in change of blood flow due to movement of the measurement site, which occurs even when the contact state between the PPG sensor and the measurement site is good; and
(4) Variation in light absorption amount due to deformation of the subcutaneous tissue associated with movement of finger or wrist.

A pseudo peak signal is mixed into the pulse wave signal due to a combination of the four factors, and it is difficult to determine which peak is the actual peak signal derived from the heartbeat or the pseudo peak signal, which decreases the accuracy of the estimation of the heart rate.

So far, various methods for reducing body motion noise (for example, an adaptive filter, frequency analysis, blind signal separation method, and the like) have been proposed (see, for example, PTLs 1 and 2). Generally, as preprocessing of the noise reduction method, a band pass filtering process for limiting the band of the pulse wave signal is applied to the pulse wave signal, and the noise reduction method is further applied to the pulse wave signal after the band pass filtering process.

### CITATION LIST

### Patent Literature

PTL 1: JP 2021-145930A
PTL 2: JP 2021-503309A

### SUMMARY OF INVENTION

### Technical Problem

However, even in a case where the peak interval is correctly detected in the pulse wave signal after the noise reduction process, an error from the heart rate interval calculated from the electrocardiograph as a reference increases. This is because the signal-to-noise ratio (S/N) of the input signal is poor and the peak position after the filtering process deviates even when the noise reduction process operates as expected. For this reason, even when the heart rate variability is analyzed using the time series data of the peak interval (heart rate) of the pulse wave signal, it greatly deviates from the result of the analysis of the heart rate variability by the electrocardiograph as a reference, and the accuracy of the healthcare application utilizing the heart rate variability decreases.

The present technology has been made in view of such a situation, and enables estimation of a heart rate with high accuracy.

### Solution to Problem

A signal processing device according to an aspect of the present technology includes a first signal quality estimator that estimates a first signal quality of an input biometric signal, a second signal quality estimator that estimates a second signal quality estimation of an output signal from a noise reduction processor, and a heart rate estimator that estimates a heart rate based on the output signal from the noise reduction processor and estimates a reliability of the estimated heart rate based on a result of the estimated first signal quality and a result of the estimated second signal quality.

In an aspect of the present technology, the estimated first signal quality is made of an input biometric signal, the estimated second signal quality is made of the output signal from a noise reduction processor, a heart rate is estimated based on the output signal from the noise reduction processor, and a reliability of the estimated heart rate is based on a result of the estimated first signal quality and a result of the estimated second signal quality. Then, the heart rate is corrected based on the basis of the estimated reliability of the estimated heart rate.

A learning device according to another aspect of the present technology comprises a first signal quality estimator that estimates a first signal quality of an input biometric signal, a second signal quality estimator that estimates a second signal quality of an output signal from a noise reduction processor, a heart rate estimator that estimates a heart rate based on the output signal from the noise reduction processor and estimates a reliability of the heart rate based on a result of the estimated first signal quality and a result of the estimated second signal quality, a correction processor that corrects the heart rate based on the estimated reliability of the estimated heart rate, and an estimation model learning unit that learns an estimation model for estimating a person's emotional state using the estimated heart rate and the estimated reliability of the estimated heart rate.

In another aspect of the present technology, a first signal quality estimation is made of an input biometric signal, a second signal quality estimation is made of an output signal from a noise reduction processor, a heart rate is estimated based on the output signal from the noise reduction processor, and a reliability of the estimated heart rate is estimated based on a result of the estimated first signal quality and a result of the estimated second signal quality. Then, the heart rate is corrected based on the estimated reliability of estimated the heart rate, and an estimation model learning unit learns an estimation model for estimating a person's emotional state using the estimated heart rate and the estimated reliability of the estimated heart rate.

### BRIEF DESCRIPTION OF DRAWINGS

FIG. 1 is a block diagram illustrating a configuration example of a wearable heart rate meter according to the first embodiment of the present technology.

FIG. 2 is a block diagram illustrating a configuration example of a signal quality estimation unit (also referred to herein as a signal quality estimator) in FIG. 1.

FIG. 3 is a block diagram illustrating a configuration example of a signal quality estimation model learning unit.

FIG. 4 is a block diagram illustrating a configuration example of a signal quality label generation unit (also referred to herein as a signal quality label generator).

FIG. 5 is a diagram illustrating an example of a pulse wave signal.

FIG. 6 is a diagram illustrating a maximum correlation coefficient of a lag having a maximum correlation.

FIG. 7 is a diagram illustrating a lag at which a correlation is maximized over time.

FIG. 8 is a flowchart illustrating a process for estimating a heart rate from a signal from a wearable heart rate meter of FIG. 1.

FIGS. 9A and 9B illustrate examples of the reliability of a final output heart rate.

FIG. 10 is a block diagram illustrating an example of a wearable heart rate meter according to a second embodiment of the present technology.

FIG. 11 is a flowchart illustrating a process of estimating a heart rate from a signal from the wearable heart rate meter in FIG. 10.

FIG. 12 is a flowchart illustrating a second process of estimating a heart rate from a signal from the wearable heart rate meter of FIG. 10.

FIG. 13 is a block diagram illustrating an example configuration of a device for learning an arousal estimation model.

FIG. 14 is a block diagram illustrating an example configuration of an arousal estimator.

FIG. 15 is a block diagram illustrating a configuration example of a computer.

### DESCRIPTION OF EMBODIMENTS

Hereinafter, modes for carrying out the present technology will be described. The description will be given in the following order.
1. First Embodiment
2. Second Embodiment
3. Third Embodiment
4. Others

### 1. First Embodiment

### <Configuration Example of Wearable Heart Rate Meter>

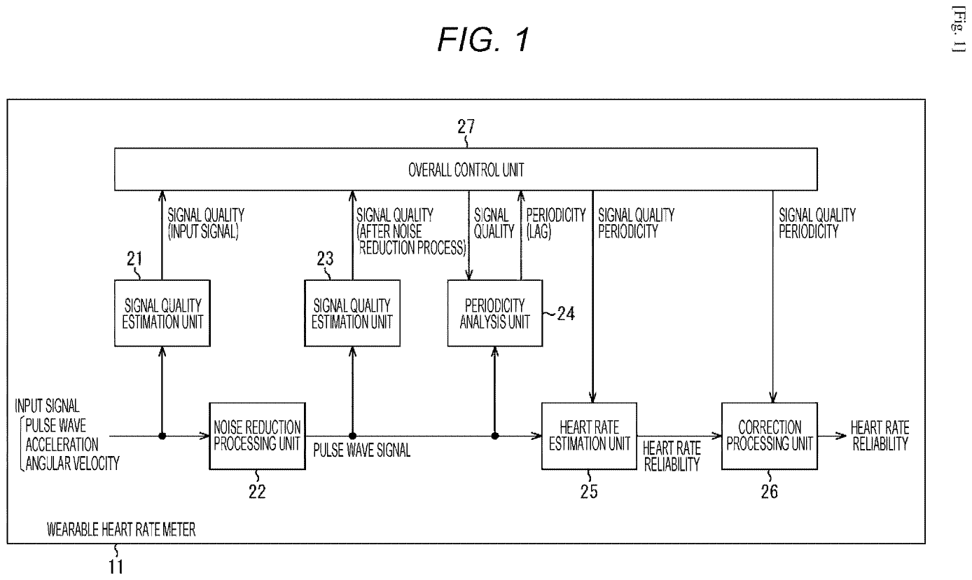

FIG. 1 is a block diagram illustrating an example configuration of a wearable heart rate meter according to the first embodiment of the present technology.

A wearable heart rate meter 11 of FIG. 1 may be included in a smart watch or the like equipped with a heart rate measurement function of a PPG method. In the case of FIG. 1, a measurement site of the wearable heart rate meter 11 may be a wrist.

Note that the measurement site is not limited to the wrist, and for example, in a case where the wearable heart rate meter 11 is a canal type headphone or a headband, the ear may be a measurement site. In a case where the wearable heart rate meter 11 is a pair of virtual reality (VR) goggles, the forehead may be a measurement site. In a case where the wearable heart rate meter 11 is a band type device, an arm or a foot on which the band is attached may be a measurement site. In a case where the wearable heart rate meter 11 is a patch-shaped device, the chest may be a measurement site.

In FIG. 1, the wearable heart rate meter 11 includes a signal quality estimation unit (also referred to herein as a signal quality estimator) 21, a noise reduction processing unit (also referred to herein as a noise reduction processor) 22, a signal quality estimation unit (also referred to herein as a signal quality estimator) 23, a periodicity analysis unit (also referred to herein as a periodicity analyzer) 24, a heart rate estimation unit (also referred to herein as a heart rate estimator) 25, a correction processing unit (also referred to herein as a correction processor) 26, and an overall control unit (also referred to herein as an overall controller) 27.

In the wearable heart rate meter 11, for example, the heart rate is measured by observing a pulse wave signal among the biometric signals.

A pulse wave signal, an acceleration signal, and an angular velocity (gyro) signal may be measured by the wearable heart rate meter 11 of FIG. 1. The pulse wave signal, the acceleration signal, and the angular velocity signal are supplied to the signal quality estimation unit 21 and the noise reduction processing unit 22.

The signal quality estimation unit 21 estimates signal quality of a pulse wave signal supplied from the heart rate meter 11 in the preceding stage (not illustrated), and supplies information indicating the estimated signal quality to the overall control unit 27.

Note that, in general, in the estimation of the signal quality of the pulse wave signal, an S/N value is used as a heart rate band (S component) and other bands (N component). Since the heart rate band of the observed pulse wave signal is unknown, the heart rate band estimated from the pulse wave signal is used. However, in a case where body motion noise is mixed into the pulse wave signal, it is difficult to estimate a correct heart rate band.

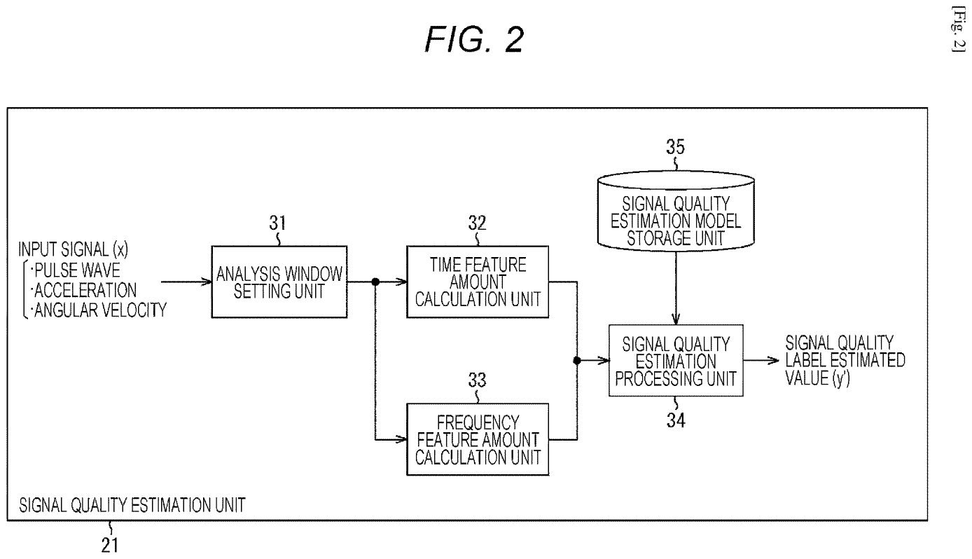

In the signal quality estimation unit 21, a signal quality estimation (DNN) model constructed by machine learning using data sets of pulse wave signals of various S/N values generated in advance is used. The signal quality estimation model is a model that estimates signal quality labels (for example, two classes of good or bad). The signal quality label is defined by comparing the calculated heart rate with the heart rate measured by the electrocardiograph and determining whether or not a difference between them is equal to or less than a preset error. Details of the signal quality estimation unit 21 will be described later with reference to FIG. 2.

The signal quality estimation unit 21 supplies information (a signal quality label and an estimated value) indicating the signal quality of the pulse wave signal to the overall control unit 27.

The noise reduction processing unit 22 reduces noise superimposed on the pulse wave signal from the pulse wave signal, the acceleration signal, and the angular velocity signal supplied from the previous stage. In the noise reduction processing unit 22, for example, noise reduction by an adaptive filter using an acceleration signal and an angular velocity signal as noise reference signals is used. The noise reduction processing unit 22 outputs the pulse wave signal after the noise reduction process to the signal quality estimation unit 23, the periodicity analysis unit 24, and the heart rate estimation unit 25.

Note that although various methods have been proposed as the noise reduction method, the present technology is a framework that does not depend on the noise reduction process, so that any noise reduction method may be used in the noise reduction processing unit 22.

The signal quality estimation unit 23 estimates the signal quality of the output signal from the noise reduction processing unit 22.

Note that although various noise reduction processes have been proposed as described above, it is difficult to completely separate noise, and thus the pulse wave signal after the noise reduction process may include residual noise.

For example, in a case where the frequency of the body motion is constant when the noise reduction process is performed by the adaptive filter, noise reduction with high accuracy can be performed. However, in a case where the body motion is an aperiodic and/or a single motion, it may be impossible to obtain an optimal filter coefficient, and it may be impossible to obtain complete noise reduction, and residual noise occurs.

Therefore, the signal quality estimation unit 23 estimates the signal quality of the pulse wave signal after the noise reduction process (also referred to herein as the output signal from the noise reduction processing unit) in order to evaluate the amount of residual noise. At this time, the signal quality estimation model may be used.

The signal quality estimation unit 23 supplies information (a signal quality label and an estimated value) indicating the signal quality of the pulse wave signal after the noise reduction process to the overall control unit 27.

The periodicity analysis unit 24 analyzes the periodicity of the output signal from the noise reduction processing unit 22 with reference to the information indicating the signal quality (before and after the noise reduction process) supplied from the overall control unit 27. Since the heart rate has high periodicity, the periodicity analysis unit 24 determines whether or not the periodicity of the pulse wave signal is high by using the characteristic that the periodicity of the heart rate derived pulse wave signal is high. The periodicity analysis unit 24 outputs the analysis result of the periodicity to the overall control unit 27.

For example, the periodicity analysis unit 24 performs a periodicity analysis using autocorrelation, and detects a lag at which the correlation is maximized. The periodicity analysis unit 24 determines that there is periodicity (high) in a case where the maximum correlation coefficient is larger than a preset threshold value, and determines that there is no periodicity (low) in a case where the maximum correlation coefficient is smaller than the preset threshold value.

Furthermore, as another example of the periodicity analysis, a lag with the maximum correlation may be stored, and it may be determined that there is periodicity in a case where the difference in the time series variation between the current lag and the past lag is smaller than a preset threshold value, and it may be determined that there is no periodicity in a case where the difference is smaller than the threshold value.

The heart rate estimation unit 25 estimates the heart rate from the output signal from the noise reduction processing unit 22 with reference to the analysis result of the periodicity supplied from the overall control unit 27.

The heart rate estimation unit 25 detects a peak of the pulse wave signal. The heart rate estimation unit 25 detects a peak of the pulse wave signal, and estimates a heart rate from a peak interval which is an interval between the detected peaks. Although various methods have been proposed as the peak detection method, the present technology is a framework that does not depend on the peak detection method, so that any peak detection method may be used in the heart rate estimation unit 25.

For example, the heart rate estimation unit 25 detects the peak interval from the maximum value detection, and uses the detected maximum value as the peak position when the peak interval is within the heart rate band of a human. Furthermore, for example, the heart rate estimation unit 25 may use, as a candidate for the peak interval, a peak position at which the peak interval and the value of the lag detected by the periodicity analysis unit 24 are close.

Furthermore, the heart rate estimation unit 25 estimates the reliability of the heart rate at the peak position on the basis of the signal quality estimation results by the signal quality estimation unit 21 and the signal quality estimation unit 23. Signal quality before and after the noise reduction process is referred to for estimation of the reliability of the heart rate. For example, in a case where the signal quality labels of the signal quality estimation unit 21 and the signal quality estimation unit 23 at the peak position are both good (high quality), it is estimated that the reliability of the heart rate at the peak position is high.

The present technology estimates the signal quality of the peak of the pulse wave signal for each heartbeat to detect the heart rate from the pulse wave signal.

That is, in the present technology, when the reliability of the heart rate at the peak position is estimated in the detection of the peak, it is not determined whether or not the peak intensity or the peak interval is the intensity or band derived from heartbeat, but it is determined whether or not the signal waveform of one beat (each heartbeat) is derived from the heartbeat, that is, whether or not the reliability is high. With this arrangement, the reliability of the heart rate at the peak position can be estimated with high accuracy.

Note that the heart rate estimation unit 25 may determine the reliability of the heart rate in consideration of the result of the periodicity analysis. With this arrangement, it is possible to determine whether or not the periodicity is a periodicity derived from the heartbeat, that is, whether or not the reliability of the periodicity is high, in addition to the signal quality of the waveform, and thus, it is possible to estimate the reliability of the heart rate with higher accuracy.

The heart rate estimation unit 25 outputs the estimated heart rate and the estimated reliability of the heart rate to the correction processing unit 26.

The correction processing unit 26 performs a correction process on the heart rate supplied from the heart rate estimation unit 25 on the basis of the reliability of the heart rate supplied from the heart rate estimation unit 25. For example, the correction processing unit 26 treats heart rate data whose reliability is less than or equal to a preset threshold value (that is, the reliability is low) as a missing value, and compensates the missing value using data with high reliability in the vicinity (past or future).

Specifically, for example, the heart rate data with low reliability is compensated using a pre-hold with the temporally closest heart rate in time with high reliability or a linear interpolation from nearby heart rates with high reliability. Since the heart rate with low reliability is the heart rate calculated from the pulse wave signal on which the body motion noise may be superimposed, the falsely detected heart rate is rejected, and the accuracy of the final output heart rate is improved.

Note that the correction processing unit 26 may change the type of correction process according to the degree of signal quality.

The correction processing unit 26 outputs the heart rate and the reliability of the heart rate that were subjected to the correction process in a subsequent stage (not illustrated).

The overall control unit 27 controls data exchange between the units.

### <Configuration of Signal Quality Estimation Unit>

FIG. 2 is a block diagram illustrating an example configuration of the signal quality estimation unit 21 in FIG. 1.

In FIG. 2, the signal quality estimation unit 21 includes an analysis window setting unit 31, a time feature amount calculation unit 32, a frequency feature amount calculation unit 33, a signal quality estimation processing unit 34, and a signal quality estimation model storage unit 35.

A pulse wave signal, an acceleration signal, and an angular velocity signal are input to the analysis window setting unit 31. In general, the feature amount is extracted by an analysis window (sliding window) of about several seconds. The analysis window setting unit 31 sets an analysis window such as four seconds for the input signal to output information regarding the set analysis window to the time feature amount calculation unit 32 and the frequency feature amount calculation unit 33.

The time feature amount calculation unit 32 calculates a feature amount of a time component in the analysis window on the basis of the information regarding the analysis window supplied from the analysis window setting unit 31 to output the calculated feature amount of the time component to the signal quality estimation processing unit 34.

The frequency feature amount calculation unit 33 calculates the feature amount of the frequency component in the analysis window on the basis of the information regarding the analysis window supplied from the analysis window setting unit 31 to output the calculated feature amount of the frequency component to the signal quality estimation processing unit 34.

The signal quality estimation processing unit 34 receives the feature amount of the time component in the analysis window supplied from the time feature amount calculation unit 32 and the feature amount of the frequency component in the analysis window supplied from the frequency feature amount calculation unit 33, estimates signal quality using the signal quality estimation model, and outputs a signal quality label and an estimated value.

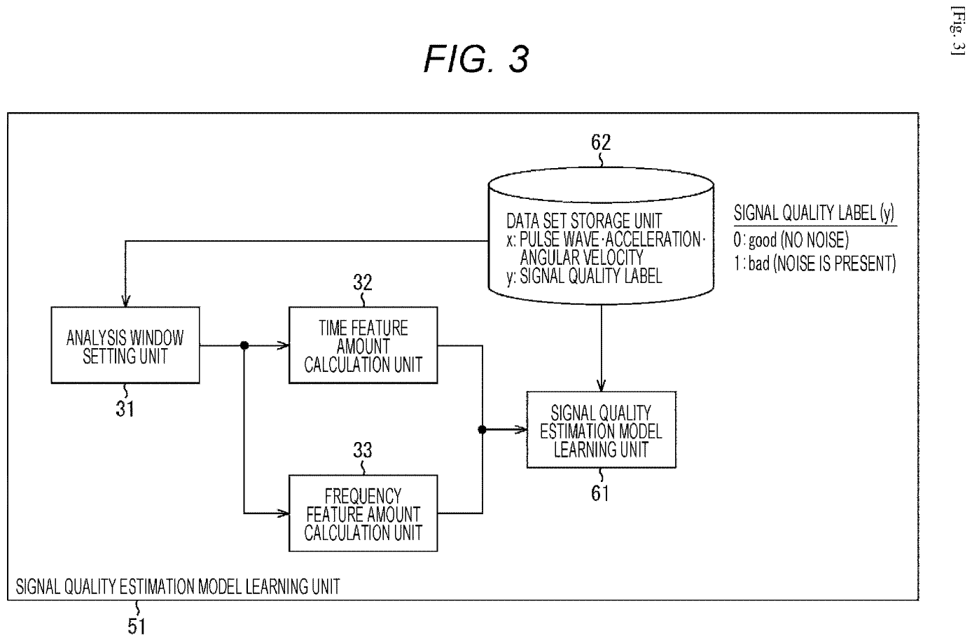

The signal quality estimation model storage unit 35 stores a signal quality estimation model used by the signal quality estimation processing unit 34. The signal quality estimation model is learned by a signal quality estimation model learning unit 51 described later with reference to FIG. 3 and stored in the signal quality estimation model storage unit 35.

### <Signal Quality Estimation Model Learning Unit>

FIG. 3 is a block diagram illustrating an example configuration of the signal quality estimation model learning unit 51.

The signal quality estimation model learning unit 51 in FIG. 3 learns the signal quality estimation model stored in the signal quality estimation model storage unit 35. The signal quality estimation model learning unit 51 may be included in the wearable heart rate meter 11 or may be included in another signal processing device. Note that, in FIG. 3, parts corresponding to those in FIG. 2 are denoted by the same reference numerals, and the description thereof will be omitted because it is redundant.

The signal quality estimation model learning unit 51 includes the analysis window setting unit 31, the time feature amount calculation unit 32, the frequency feature amount calculation unit 33, a signal quality estimation model learning unit 61, and a data set storage unit 62.

A data set of the pulse wave signal, the acceleration signal, and the angular velocity signal (x) and the signal quality label (y) is stored in the data set storage unit 62 is input to the analysis window setting unit 31 in FIG. 3.

The signal quality estimation model learning unit 61 learns the signal quality estimation model by using a signal quality label defined in advance for the data set of the data set storage unit 62 with the feature amount of the time component in the analysis window supplied from the time feature amount calculation unit 32 and the feature amount of the frequency component in the analysis window supplied from the frequency feature amount calculation unit 33 as inputs. The learned signal quality estimation model is used in the signal quality estimation units 21 and 23 in FIG. 1 and the like.

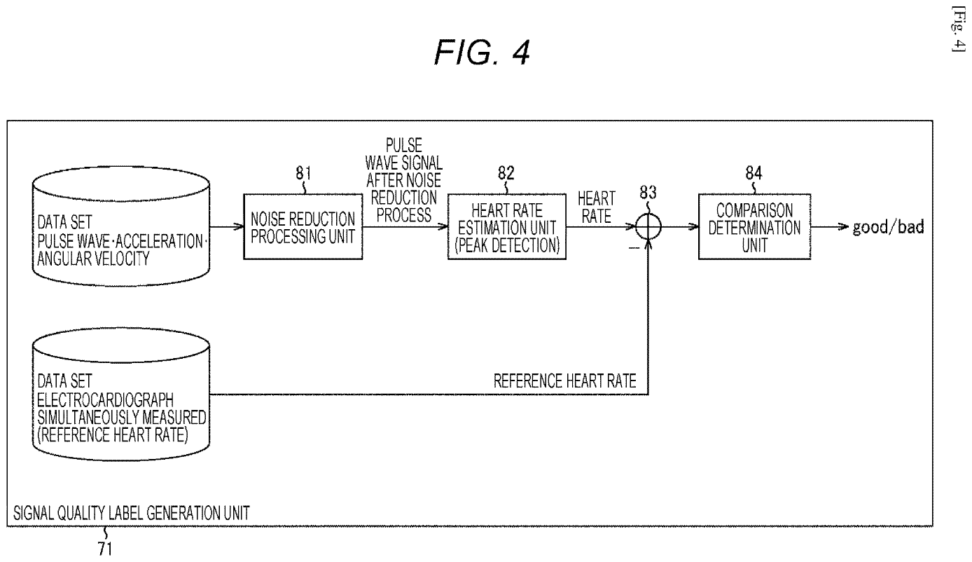

The data set storage unit 62 stores a data set of a pulse wave signal, an acceleration signal, and an angular velocity signal (x) and a signal quality label (y), and a signal quality label (for example, two classes of good or bad) defined in advance for each data set. The signal quality label is defined by a signal quality label generation unit (also referred to herein as a signal quality label generator) 71 described later with reference to FIG. 4 and stored in the data set storage unit 62.

### <Signal Quality Label Generation Unit>

FIG. 4 is a block diagram illustrating an example configuration of the signal quality label generation unit 71.

For example, data sets of pulse wave signals having various S/N values are constructed in advance by performing various operations at the time of simultaneous measurement using the electrocardiograph and the wearable heart rate meter 11.

The data set of the pulse wave signal includes the pulse wave signal, the acceleration signal, and the angular velocity signal measured by the wearable heart rate meter 11, and the heart rate measured by the electrocardiograph.

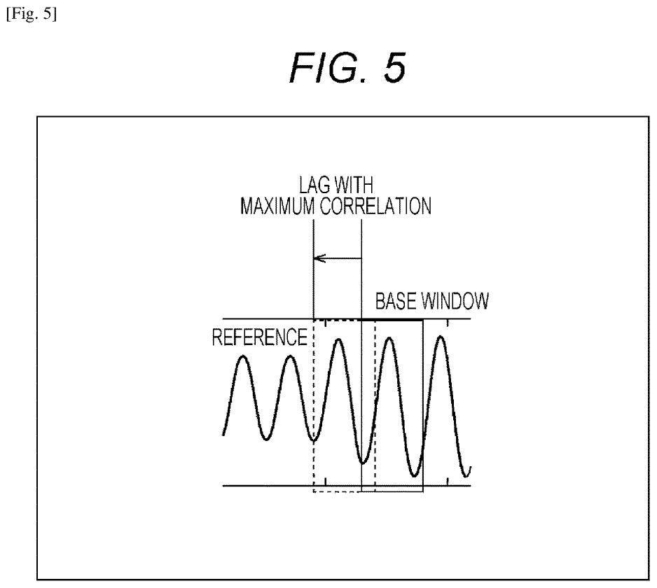

The signal quality label generation unit 71 of FIG. 4 defines a signal quality label for each data set and stores the defined signal quality label with each respective data set in the data set storage unit of FIG. 5. The signal quality label generation unit 71 may be included in the wearable heart rate meter 11 or may be included in another signal processing device.

The signal quality label generation unit 71 includes a noise reduction processing unit 81, a heart rate estimation unit 82, an arithmetic unit 83, and a comparison determination unit 84.

The pulse wave signal, the acceleration signal, and the angular velocity signal of the data set are input to the noise reduction processing unit 81. The noise reduction processing unit 81 performs a noise reduction process on the pulse wave signal and outputs the signal to the heart rate estimation unit 82.

The heart rate estimation unit 82 detects a peak, a peak interval, and the like from the pulse wave signal after the noise reduction process, and estimates the heart rate from the detected peak interval. The heart rate estimation unit 82 outputs the estimated heart rate to the arithmetic unit 83.

The heart rate estimated by the heart rate estimation unit 82 and the reference heart rate of the data set are supplied to the arithmetic unit 83. The arithmetic unit 83 outputs the difference between the heart rate estimated by the heart rate estimation unit 82 and the reference heart rate to the comparison determination unit 84.

The comparison determination unit 84 defines a signal quality label (for example, two classes of good or bad) on the basis of whether or not the difference supplied from the arithmetic unit 83 is equal to or less than a preset error. That is, in a case where the difference is equal to or less than the preset error, the signal quality label is defined in the class of good; in a case where the difference is greater than the preset error, the signal quality label is defined in the class of bad.

Next, an example of the periodicity analysis using autocorrelation by the periodicity analysis unit 24 will be described.

FIG. 5 is a diagram illustrating an example of a pulse wave signal.

In FIG. 5, the vertical axis represents a pulse wave, and the horizontal axis represents time. FIG. 5 illustrates a base window (solid line) set by the periodicity analysis unit 24 and a reference window (broken line) set at a position shifted from a position of the base window in the past direction.

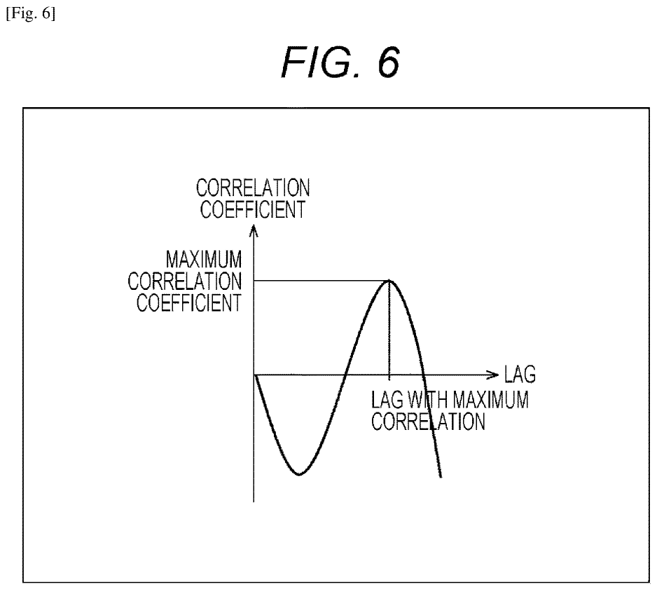

FIG. 6 is a diagram illustrating correlation coefficients of the lags having the maximum correlation.

In FIG. 6, the vertical axis represents the correlation coefficient, and the horizontal axis represents the lag.

As illustrated in FIG. 5, the periodicity analysis unit 24 sets a base window for the pulse wave signal, and sets a reference window at a position where the lag is shifted in the past direction. Then, the correlation coefficient between the pulse wave signal of the set base window and the pulse wave signal of the reference window is calculated, whereby a lag with the maximum correlation is detected as illustrated in FIG. 6.

Note that, in a case where the correlation coefficient (that is, the maximum correlation coefficient) of the lag having the maximum correlation is smaller than a preset threshold value, the periodicity analysis unit 24 determines that the pulse wave signal has no periodicity (low).

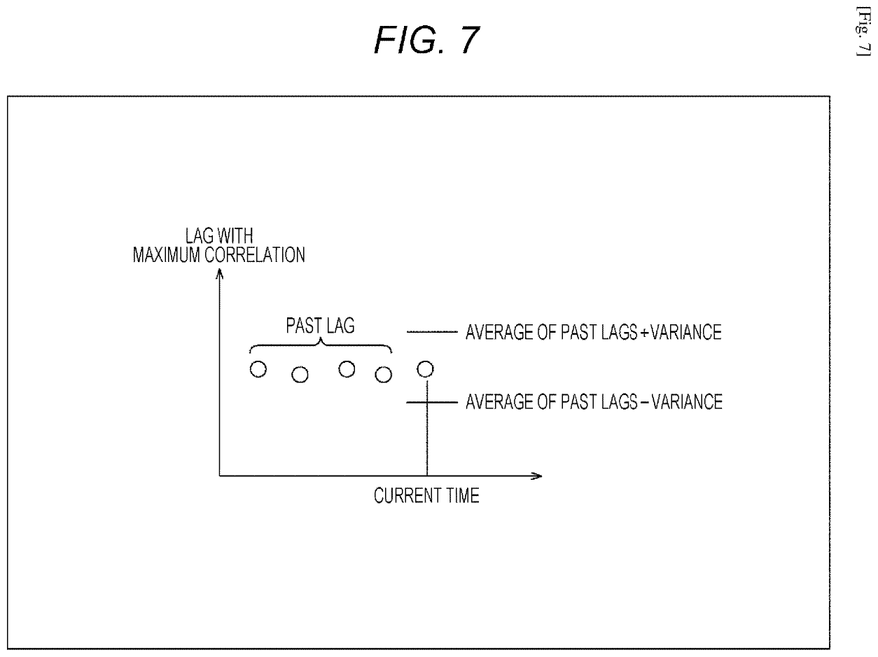

Furthermore, the examples of FIGS. 5 and 6 are examples, and the periodicity analysis unit 24 may perform a periodicity analysis as described later with reference to FIG. 7.

FIG. 7 is a diagram illustrating the lag with the maximum correlation over time.

In FIG. 7, the lag with the maximum correlation is illustrated over a period of time. For example, the lag calculated at the current time is included within the range of the average±standard deviation of the lags (data) having the maximum correlation in the past.

As illustrated in FIG. 7, the periodicity analysis unit 24 may determine that there is periodicity in a case where the lag calculated at the current time is included within the range of the average±standard deviation of the lags having the maximum correlation in the past, and may determine that there is no periodicity in a case where the lag is not included within the range of the average±standard deviation of the lags having the maximum correlation in the past.

### <Process of Wearable Heart Rate Meter>

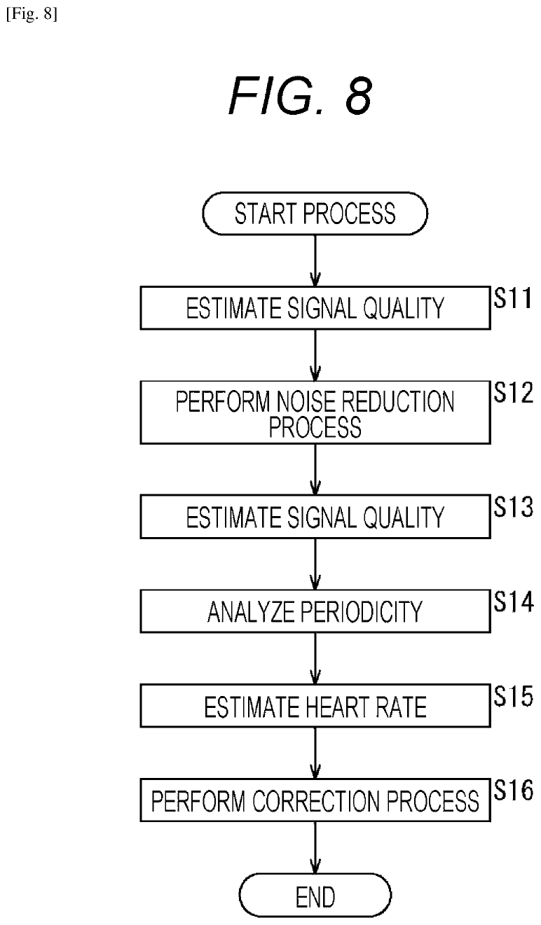

FIG. 8 is a flowchart illustrating a process of the wearable heart rate meter 11 of FIG. 1.

The pulse wave signal, the acceleration signal, and the angular velocity signal are supplied to the signal quality estimation unit 21 and the noise reduction processing unit 22.

In step S11, the signal quality estimation unit 21 estimates the signal quality of the pulse wave signal supplied from the previous stage to output information (a signal quality label and an estimated value) indicating the signal quality to the overall control unit 27.

In step S12, the noise reduction processing unit 22 reduces noise superimposed on the pulse wave signal, the acceleration signal, and the angular velocity signal supplied from the previous stage. The noise reduction processing unit 22 outputs the pulse wave signal after the noise reduction process to the signal quality estimation unit 23, the periodicity analysis unit 24, and the heart rate estimation unit 25.

In step S13, the signal quality estimation unit 23 estimates the signal quality of the pulse wave signal after the noise reduction process supplied from the noise reduction processing unit 22. The signal quality estimation unit 23 supplies information (a signal quality label and an estimated value) indicating the signal quality of the pulse wave signal after the noise reduction process to the overall control unit 27.

In step S14, the periodicity analysis unit 24 analyzes the periodicity of the pulse wave signal after the noise reduction process supplied from the noise reduction processing unit 22 with reference to the information indicating the signal quality supplied from the overall control unit 27. The periodicity analysis unit 24 outputs the analysis result of the periodicity to the overall control unit 27.

In step S15, the heart rate estimation unit 25 estimates the heart rate from the pulse wave signal after the noise reduction process supplied from the noise reduction processing unit 22 with reference to the analysis result of the periodicity supplied from the overall control unit 27. Furthermore, the heart rate estimation unit 25 estimates the reliability of the heart rate at the peak position on the basis of the signal quality estimation results of the signal quality estimation unit 21 and the signal quality estimation unit 23. At this time, as described above, the analysis result of the periodicity supplied from the overall control unit 27 may be referred to. The heart rate estimation unit 25 outputs the estimated heart rate and the estimated reliability of the heart rate to the correction processing unit 26.

In step S16, the correction processing unit 26 performs a correction process on the estimated heart rate supplied from the heart rate estimation unit 25 on the basis of the estimated reliability of the heart rate supplied from the heart rate estimation unit 25. The correction processing unit 26 outputs the heart rate and the reliability of the heart rate that were subjected to the correction process to a subsequent stage (not illustrated).

Here, in general, outlier detection is performed from statistical information about heart rate time series data, linear prediction, or the like. However, the heart rate greatly fluctuates due to the influence of the autonomic nerve state of the user or the like, and may be erroneously detected as an outlier.

On the other hand, in the present technology, by using the reliability of determining whether or not the waveform shapes of the input pulse wave signal and the pulse wave signal after the noise reduction process are derived from the heartbeat, the pulse rate is not erroneously detected as an outlier, and the outlier can be detected, so that the heart rate can be estimated with high accuracy.

Note that the reliability of the final heart rate output from the correction processing unit 26 is not limited to the signal quality label of good or bad in the signal quality estimation results of the signal quality estimation units 21 and 23.

### <Example of Reliability of the Final Heart Rate Output>

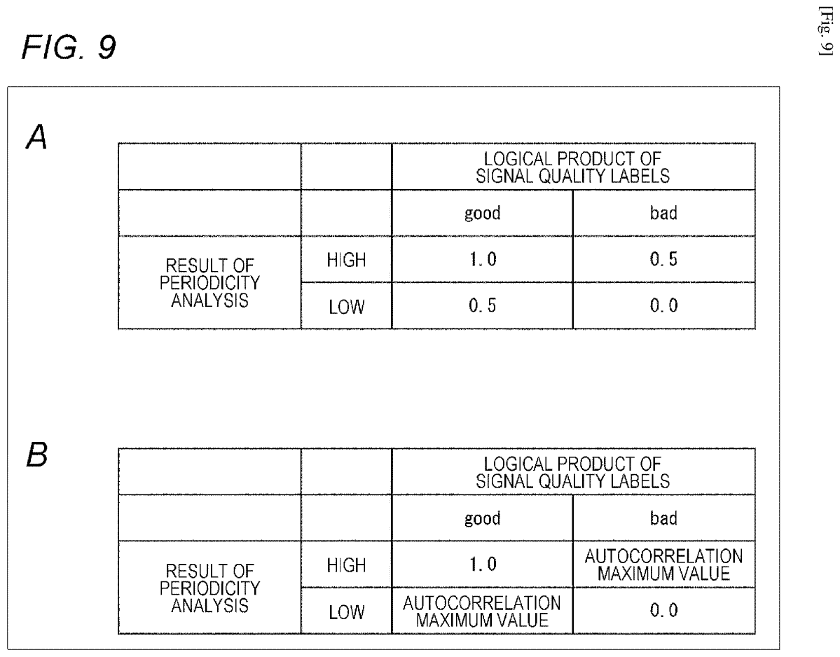

FIGS. 9A and 9B illustrate examples of a reliability of a final output heart rate.

FIG. 9A illustrates a case where the logical product of the signal quality labels in the signal quality estimation results of the signal quality estimation units 21 and 23 is good and the result of the periodicity analysis of the periodicity analysis unit 24 is high, the reliability of the final output heart rate is 1.0.

Note that the logical product of the signal quality labels in the signal quality estimation results of the signal quality estimation units 21 and 23 represents good only in a case where the signal quality label in the signal quality estimation result of the signal quality estimation unit 21 is good and the signal quality label in the signal quality estimation result of the signal quality estimation unit 23 is good.

In a case where the logical product of the signal quality labels in the signal quality estimation results of the signal quality estimation units 21 and 23 is good and the result of the periodicity analysis of the periodicity analysis unit 24 is low, it is indicated that the reliability of the final output heart rate is 0.5.

In a case where the logical product of the signal quality labels in the signal quality estimation results of the signal quality estimation units 21 and 23 is bad and the result of the periodicity analysis of the periodicity analysis unit 24 is high, it is indicated that the reliability of the final output heart rate is 0.5.

In a case where the logical product of the signal quality labels in the signal quality estimation results of the signal quality estimation units 21 and 23 is bad and the result of the periodicity analysis of the periodicity analysis unit 24 is low, it is indicated that the reliability of the final output heart rate is 0.0.

FIG. 9B illustrates a case where the logical product of the signal quality labels in the signal quality estimation results of the signal quality estimation units 21 and 23 is good and the result of the periodicity analysis of the periodicity analysis unit 24 is high, the reliability of the final output heart rate is 1.0.

In a case where the logical product of the signal quality labels in the signal quality estimation results of the signal quality estimation units 21 and 23 is good and the result of the periodicity analysis of the periodicity analysis unit 24 is low, it is indicated that the reliability of the final output heart rate is the autocorrelation maximum value (correlation maximum coefficient).

In a case where the logical product of the signal quality labels in the signal quality estimation results of the signal quality estimation units 21 and 23 is bad and the result of the periodicity analysis of the periodicity analysis unit 24 is high, it is indicated that the reliability of the final output heart rate is the autocorrelation maximum value.

In a case where the logical product of the signal quality labels in the signal quality estimation results of the signal quality estimation units 21 and 23 is bad and the result of the periodicity analysis of the periodicity analysis unit 24 is low, it is indicated that the reliability of the final output heart rate is 0.0.

As described above, as the reliability of the final outputted heart rate from the correction processing unit 26, not only the signal quality label of good or bad in the signal quality estimation results of the signal quality estimation units 21 and 23 but also values based on the results of the signal quality estimation units 21 and 23 and the periodicity analysis unit 24 may be output.

### 2. Second Embodiment

### <Configuration Example of Wearable Heart Rate Meter>

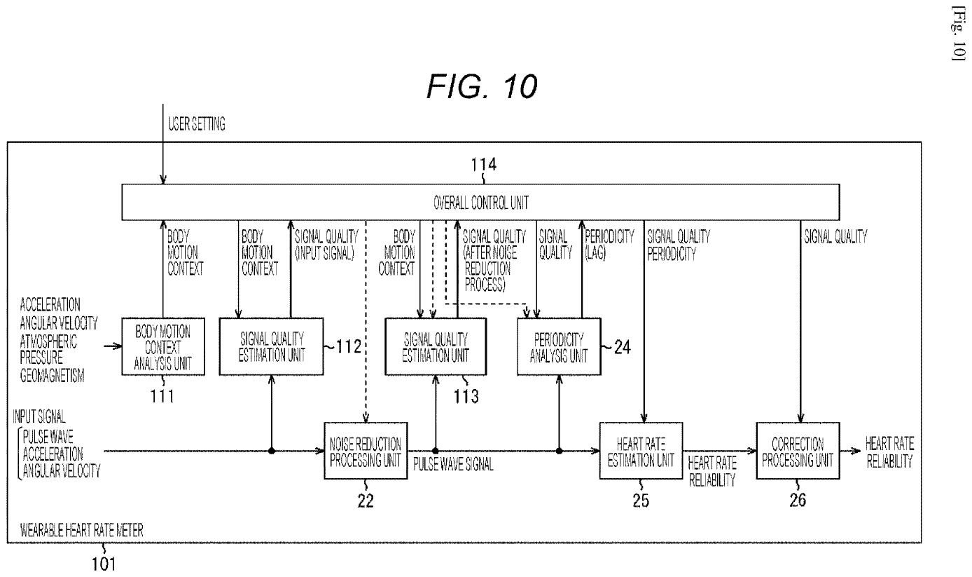

FIG. 10 is a block diagram illustrating an example configuration of a wearable heart rate meter according to the second embodiment of the present technology.

A wearable heart rate meter 101 of FIG. 10 differs from the wearable heart rate meter 11 of FIG. 1 in that a body motion context analysis unit (also referred to herein as a body motion context analyzer) 111 is added; and that the signal quality estimation unit 21, the signal quality estimation unit 23, and the overall control unit 27 are replaced with a signal quality estimation unit 112, a signal quality estimation unit 113, and an overall control unit 114. In FIG. 10, parts corresponding to those in FIG. 1 are denoted by the same reference numerals, and the description thereof will be omitted.

The body motion context analysis unit 111 estimates (analyzes) a body motion state (sleeping, running, walking, sitting, etc.) the user is in on the basis of the body motion information about the user.

Specifically, the body motion context analysis unit 111 estimates what activity the user has been doing, a body motion context indicating a body motion state of the user on the basis of sensor information such as acceleration, angular velocity, atmospheric pressure, and geomagnetism as the body motion information about the user. The estimated body motion context is output to the overall control unit 114.

Note that although various methods have been proposed as context analysis methods, the present technology is a framework that does not depend on context analysis processing, so that any context analysis method may be used in the body motion context analysis unit 111.

The body motion context is also input to the signal quality estimation unit 112. At this time, the signal quality estimation unit 112 uses a signal quality estimation model constructed by machine learning using a data set of pulse wave signals of various S/N values generated in advance and a body motion context as inputs. The other configurations of the signal quality estimation unit 112 are similar to those of the signal quality estimation unit 21. The same may be applied to the signal quality estimation unit 113.

On the basis of the body motion context supplied from the body motion context analysis unit 111, the signal quality estimation result by the signal quality estimation unit 112, and the signal quality estimation result after the noise reduction process by the signal quality estimation unit 113, the overall control unit 114 performs control to operate or stop the processing of the noise reduction processing unit 22, the signal quality estimation unit 113, and the periodicity analysis unit 24 as indicated by dotted arrows.

For example, the body motion context analysis unit 111 estimates the presence or absence of body motion (static/dynamic determination) and the body motion intensity (magnitude) as the body motion context on the basis of a threshold value set in advance from the norm value of the acceleration sensor. The presence or absence of the body motion is determined by comparing the magnitude of the body motion with a predetermined threshold value α (α is a small value close to 0), and in a case where the magnitude of the body motion is smaller than the predetermined threshold value α, it is determined that there is no body motion.

In this case, the overall control unit 114 calculates in advance the limit performance of the noise reduction process with respect to the input signal quality and the body motion intensity on the basis of, for example, the body motion intensity estimated by the body motion context analysis unit 111, the signal quality estimation result by the signal quality estimation unit 112, and the signal quality estimation result after the noise reduction process by the signal quality estimation unit 113.

In a case where it is determined that the body motion intensity estimated by the body motion context analysis unit 111 exceeds the limit performance of the noise reduction process, the reliability estimated by the heart rate estimation unit 25 hardly changes even in a case where the processing after the noise reduction process (at least one of the noise reduction process, the signal quality estimation after the noise reduction process, or the periodicity analysis) is performed.

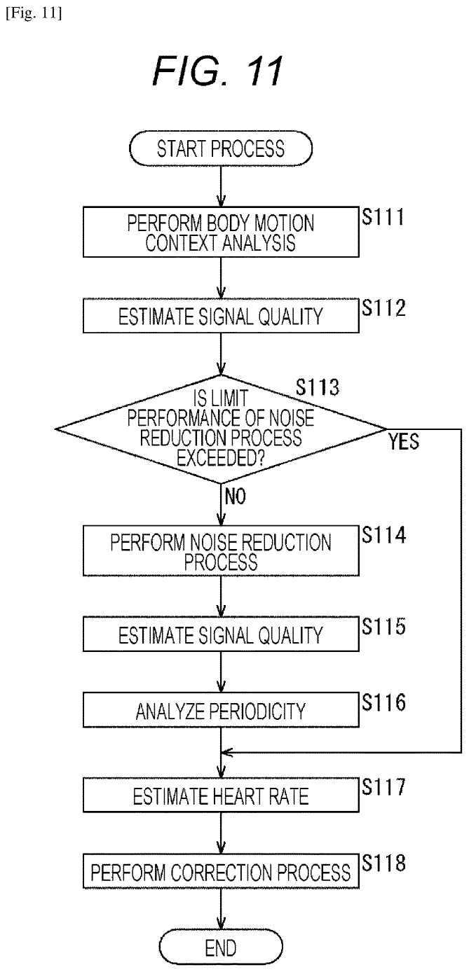

Therefore, in a case where it is determined that the body motion intensity estimated by the body motion context analysis unit 111 exceeds the limit performance of the body motion noise reduction process, the overall control unit 114 performs control to stop the above-described processes after the noise reduction process, at least the periodicity analysis process (see FIG. 11). This makes it possible to reduce the calculation cost and power consumption of the framework (processing) as a whole.

Furthermore, in a case where the body motion context analysis unit 111 estimates that the body motion is smaller than the predetermined threshold value α and the signal quality estimation unit 112 estimates that the signal quality is better (higher) than the predetermined threshold value β, it can be obviously seen that noise is hardly superimposed on the input pulse wave.

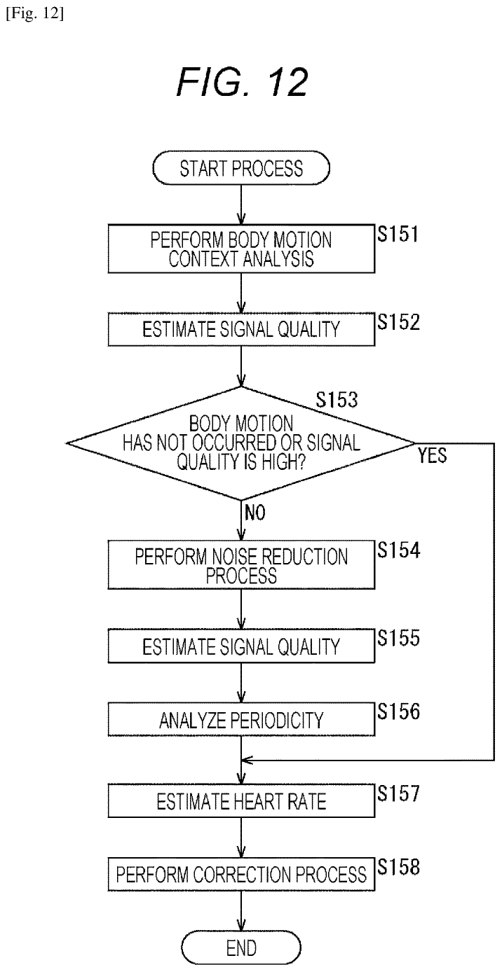

Therefore, the overall control unit 114 stops the processes of a noise reduction processing unit 12, the signal quality estimation unit 113, and the periodicity analysis unit 24 (see FIG. 12). This makes it possible to reduce the calculation cost and power consumption of the framework (processing) as a whole.

### <First Process of Wearable Heart Rate Meter>

FIG. 11 is a flowchart for explaining a first process of the wearable heart rate meter 101 in FIG. 10.

The pulse wave signal, the acceleration signal, and the angular velocity signal are supplied to the signal quality estimation unit 112 and the noise reduction processing unit 22. Furthermore, sensor information such as acceleration, angular velocity, atmospheric pressure, and geomagnetism is input to the body motion context analysis unit 111.

In step S111, the body motion context analysis unit 111 receives the sensor information as the body motion information about the user, performs the body motion context analysis, and estimates the body motion intensity. The estimated body motion intensity is output to the overall control unit 27.

In step S112, the signal quality estimation unit 112 estimates the signal quality of the pulse wave signal supplied from the previous stage to output information (a signal quality label and an estimated value) indicating the signal quality to the overall control unit 114. At this time, as described above, the body motion context supplied from the overall control unit 114 may be used as an input.

In step S113, the overall control unit 114 determines whether or not the body motion intensity supplied from the body motion context analysis unit 111 exceeds the limit performance of the noise reduction process. In a case where it is determined in step S113 that the body motion intensity does not exceed the limit performance of the noise reduction process, the process proceeds to step S114.

Since the process of steps S114 to S118 is basically similar to the process of steps S12 to S16 of FIG. 8, the description thereof will be omitted.

In a case where it is determined in step S113 that the body motion intensity exceeds the limit performance of the noise reduction process, the steps S114 to S116 are skipped, and the process proceeds to step S117. That is, since there is no meaning in performing the process, the noise reduction processing unit 22, the signal quality estimation unit 113, and the periodicity analysis unit 24 stop each process.

In a case where the steps S114 to S116 are skipped, in step S117, the heart rate estimation unit 25 estimates the heart rate from the pulse wave signal supplied from the noise reduction processing unit 22 and not subjected to the noise reduction process. Furthermore, the heart rate estimation unit 25 estimates the reliability of the heart rate at the detected peak position on the basis of the signal quality estimation result of the signal quality estimation unit 112. The heart rate estimation unit 25 outputs the estimated heart rate and the estimated reliability of the heart rate to the correction processing unit 26.

Then, in step S118, the correction processing unit 26 performs a correction process on the heart rate on the basis of the estimated reliability of the heart rate supplied from the heart rate estimation unit 25. The correction processing unit 26 outputs the heart rate and the reliability of the heart rate that were subjected to the correction process to a subsequent stage (not illustrated).

That is, even in a case where it is determined that the body motion intensity exceeds the limit performance of the noise reduction process, the steps S117 and S118 are performed, but the output has a signal quality label of bad.

As described above, since the processes of the noise reduction processing unit 22, the signal quality estimation unit 113, and the periodicity analysis unit 24 are skipped according to the body motion context, it is possible to reduce the calculation cost and the power consumption of the framework (processing) as a whole.

### <Second Process of Wearable Heart Rate Meter>

FIG. 12 is a flowchart illustrating the second process of the wearable heart rate meter 101 of FIG. 10.

The pulse wave signal, the acceleration signal, and the angular velocity signal are supplied to the signal quality estimation unit 112 and the noise reduction processing unit 22. Furthermore, sensor information such as acceleration, angular velocity, atmospheric pressure, and geomagnetism is input to the body motion context analysis unit 111.

In step S151, the body motion context analysis unit 111 receives the sensor information as the body motion information about the user, performs the body motion context analysis, and estimates the presence or absence of the body motion. The presence or absence of the estimated body motion is output to the overall control unit 114.

In step S152, the signal quality estimation unit 112 estimates the signal quality of the pulse wave signal supplied from the previous stage to output information (a signal quality label and an estimated value) indicating the signal quality to the overall control unit 114.

In step S153, the overall control unit 114 determines whether body motion has not occurred, or the signal quality is high. In a case where the body motion supplied from the body motion context analysis unit 111 is equal to or greater than the predetermined threshold value α or the signal quality is equal to or less than the predetermined threshold value β, it is determined in step S153 that the body motion has occurred or the signal quality is poor, and the process proceeds to step S154.

Since the process of steps S154 to S158 is basically similar to the process of steps S12 to S16 of FIG. 8, the description thereof will be omitted.

In a case where the body motion is smaller than the predetermined threshold value α and the signal quality is better than the predetermined threshold value β in step S153, it is determined that the body motion has not occurred and the signal quality is high, steps S154 to S156 are skipped, and the process proceeds to step S157. That is, since the signal quality is high, the noise reduction processing unit 22, the signal quality estimation unit 113, and the periodicity analysis unit 24 stop each process.

In a case where the steps S154 to S156 are skipped, in step S157, the heart rate estimation unit 25 estimates the heart rate from the pulse wave signal supplied from the noise reduction processing unit 22 and not subjected to the noise reduction process. Furthermore, the heart rate estimation unit 25 estimates the reliability of the heart rate at the detected peak position on the basis of the signal quality estimation result of the signal quality estimation unit 112. The heart rate estimation unit 25 outputs the estimated heart rate and the estimated reliability of the heart rate to the correction processing unit 26.

Then, in step S158, the correction processing unit 26 performs a correction process on the heart rate on the basis of the reliability of the heart rate supplied from the heart rate estimation unit 25. The correction processing unit 26 outputs the heart rate and the reliability of the heart rate that were subjected to the correction process to a subsequent stage (not illustrated). In this case, since the signal quality is good, the signal quality label corresponding to good or good is output.

As described above, since the processes of the noise reduction processing unit 22, the signal quality estimation unit 113, and the periodicity analysis unit 24 are skipped according to the body motion context, it is possible to reduce the calculation cost and the power consumption of the framework (processing) as a whole.

Note that, in FIGS. 11 and 12, an example is described in which the processes of the noise reduction processing unit 22, the signal quality estimation unit 113, and the periodicity analysis unit 24 are skipped, but instead of skipping all three, at least one process of the noise reduction processing unit 22, the signal quality estimation unit 113, and the periodicity analysis unit 24 may be skipped.

Furthermore, in the wearable heart rate monitor 11 of FIG. 1 and the wearable heart rate monitor 101 of FIG. 10, the correction processing unit 26 is not required, and the wearable heart rate monitor 11 of FIG. 1 and the wearable heart rate monitor 101 of FIG. 10 may be configured without the correction processing unit 26.

Note that, in the above description, the example of the pulse wave signal is described, but the present technology can be applied not only to the pulse wave but also to a biometric signal having high periodicity such as blood flow or continuous blood pressure.

### 3. Third Embodiment

The third embodiment describes an example in which the beat-by-beat heart rate (i.e., heart rate variability, hereinafter referred to as instantaneous heart rate) and the reliability of the instantaneous heart rate, which were estimated more precisely in the first and second forms described above, are used to estimate arousal level.

### <Example Configuration of a Device for Learning an Arousal Estimation Model>

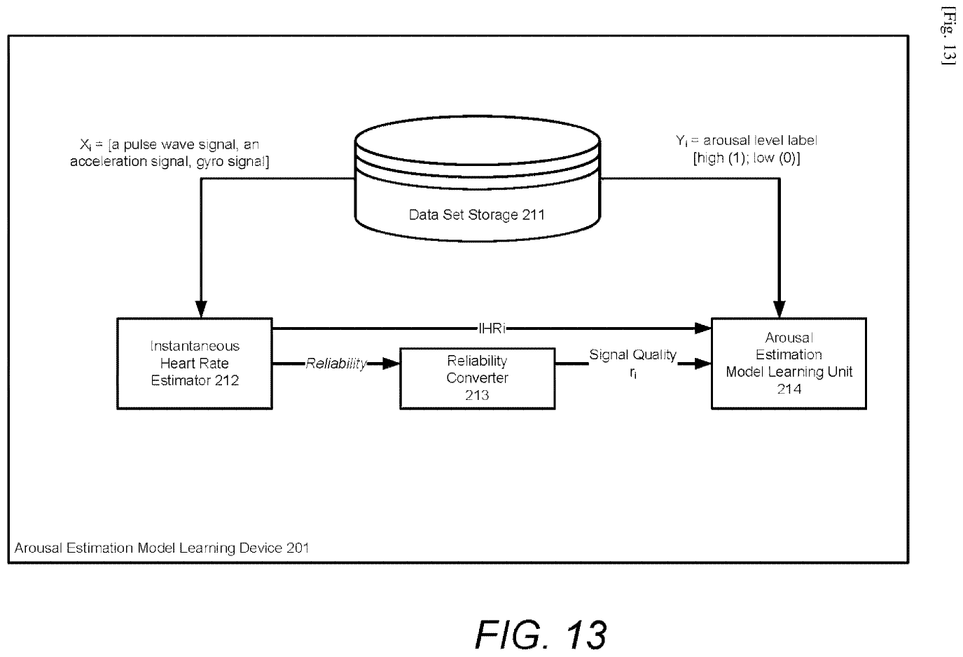

FIG. 13 is a block diagram showing an example configuration of an arousal estimation model learning device 201 for the third implementation of this technology.

The device 201 for learning an arousal estimation model shown in FIG. 13 is a learning device for learning an arousal estimation model, which is a machine learning model for estimating arousal, one of the user's emotional states, from pulse wave signals using the instantaneous heart rate and reliability level estimated from pulse wave signals in the first and second implementation forms described above.

The device 201 for learning an arousal estimation model is configured to include a data set storage unit 211, an instantaneous heart rate estimation unit 212, a reliability conversion unit 213, and an arousal estimation model learning unit 214.

The data set storage unit 211 stores n samples of the data set of Xᵢ=[pulse wave signal, acceleration signal, gyro signal] at the arousal level label (Yᵢ=high (1) or low (0)) indicating the user's arousal level status.

The instantaneous heart rate estimation unit 212 is equivalent to a device that estimates the instantaneous heart rate, such as the wearable heart rate monitor 11 in FIG. 1 or the wearable heart rate monitor 101 in FIG. 10.

That is, the instantaneous heart rate estimation unit 212 estimates the instantaneous heart rate IHRᵢ and its reliability as in FIG. 1 wearable heart rate monitor 11 or FIG. 10 wearable heart rate monitor 101, using Xᵢ supplied from the data set storage unit 211 as input. The instantaneous heart rate estimation unit 212 outputs the instantaneous heart rate IHR; to the arousal estimation model training unit 214 and the confidence level of the instantaneous heart rate reliability to the reliability conversion unit 213.

The reliability conversion unit 213 uses the reliability supplied from the instantaneous heart rate estimation unit 212 as input and uses a pre-set LUT (look-up table) or conversion function (for example, a linear function or nonlinear function such as sigmoid function) to calculate a signal quality rᵢ to be used in the wakefulness estimation model learning unit 214. The reliability conversion unit 213 calculates the signal quality rᵢ using a predefined LUT (look-up table) or conversion function (e.g., a nonlinear function such as a linear function or sigmoid function). The reliability conversion unit 213 outputs the calculated signal quality rᵢ to the arousal level estimation model learning unit 214.

The arousal estimation model learning unit 214 learns an arousal estimation model that estimates the arousal level label (0 or 1) using the instantaneous heart rate IHRᵢ supplied from the instantaneous heart rate estimation unit 212 and the signal quality rᵢ supplied from the reliability conversion unit 213.

As an example of a learning method performed in the arousal estimation model learning unit 214, a binary classification model learning method using DNN is described.

In general, the logarithmic loss function LBCE for each data used to train binary classification models does not take into account the signal quality of the data used for training, which is a cause of model accuracy degradation.

Therefore, in the arousal estimation model learning unit 214, a weighted logarithmic loss function Lₗₒₛₛ by signal quality rᵢ is used, as shown in the following equation (1), so that data with a higher signal quality make a higher contribution (to the error) when learning the model.

$$\begin{matrix}〈 Equation 1 〉 & \\ {L}_{loss} = - \frac{1}{n} {\sum }_{i = 1}^{n} {r}_{i} \cdot ( {Y}_{i} \cdot log {\hat{Y}}_{i} + ( 1 - {Y}_{i} ) \cdot log ( 1 - {\hat{Y}}_{i} ) ) & ( 1 )\end{matrix}$$

Where, Yᵢ is the true value of the arousal label; \hat{ }(hat) of Yᵢ is the predicted value (probability value) of the arousal label estimated by the model. Also, rᵢ is the signal quality (low 0.0 to high 1.0). The higher the signal quality, the higher the sample contribution during model training.

In other words, the arousal estimation model learning unit 214 repeats learning of the arousal estimation model so that the higher the quality, the more error is given, and the model coefficients are changed to minimize the error so that the cost becomes small. This makes it possible to suppress model accuracy degradation. The example described here is based on a logarithmic loss function, but is not limited to this.

The arousal level estimation model learned by the arousal level estimation model learning section 214 is used in the arousal level estimation described next.

### <Example Configuration of an Arousal Estimation Device>

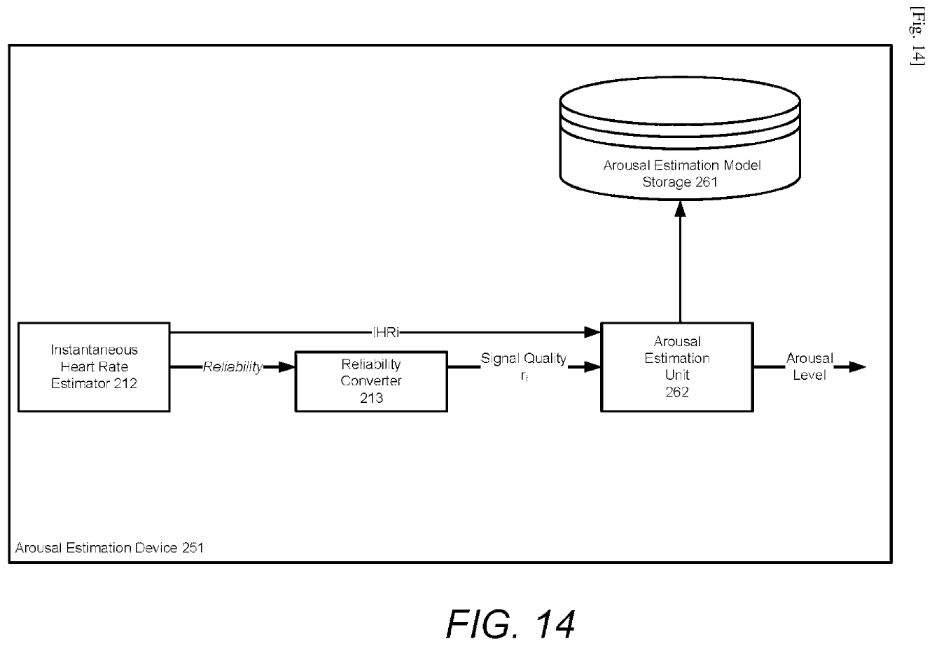

FIG. 14 is a block diagram showing an example configuration of an arousal estimation device 251 for the third implementation of this technology.

In the arousal estimation device 251 shown in FIG. 14, arousal estimation is performed using the arousal estimation model learned by the arousal estimation model learning device 201 shown in FIG. 13.

The arousal estimation device 251 is configured to include the instantaneous heart rate estimation unit 212 and the reliability conversion unit 213, the arousal estimation model storage unit 261, and the arousal estimation unit 262 of FIG. 13.

The instantaneous heart rate estimation unit 212 estimates the instantaneous heart rate IHRᵢ and its reliability as in FIG. 13, using Xᵢ supplied from sensors, for example, as input. The instantaneous heart rate estimator 212 outputs the instantaneous heart rate IHRᵢ to the arousal estimator 262, and the of the instantaneous heart rate to the reliability converter 213.

As in FIG. 13, the reliability conversion unit 213 uses the reliability supplied from the instantaneous heart rate estimation unit 212 as input and calculates the signal quality rᵢ to be used in the arousal estimation unit 262 using a pre-set LUT (look up table) and conversion function. The reliability conversion section 213 outputs the calculated signal quality r; to the arousal estimation unit 262.

The arousal estimation model storage unit 261 stores the arousal estimation model learned by the arousal estimation model learning device 201 shown in FIG. 13.

The arousal estimation unit 262 uses the instantaneous heart rate IHRᵢ supplied by the instantaneous heart rate estimation unit 212 and the signal quality rᵢ supplied by the reliability conversion unit 213 as inputs, and estimates the arousal level using the arousal estimation model read from the arousal estimation model storage unit 261.

Specifically, the arousal estimation unit 262 performs weighted prediction by the signal quality rᵢ so that data with higher signal quality will have a higher contribution in the estimation. For example, the arousal estimation unit 262 estimates the arousal level by weighted prediction with m predictions that are close in time to the input Xᵢ, as shown in the following equation (2).

$$\begin{matrix}〈 Equation 2 〉 & \\ {\hat{Y}}_{i} = {\sum }_{j}^{m} {r}_{i + j} \cdot f ( {IHR}_{i + j} ) / {\sum }_{j}^{m} {r}_{i + j} & ( 2 )\end{matrix}$$

Where i indicates the current time to be predicted. j indicates a time close to time i.

The arousal estimation unit 262 outputs the estimated arousal level to a later stage. The estimated arousal level is used in later stages for applications that require information on arousal level.

As described above, in the third implementation, the heart rate and the reliability of the heart rate, which were estimated more precisely in the first and second implementations described above, are used for the arousal estimation. This makes it possible to estimate the arousal level with higher accuracy.

In the arousal estimation model learning device 201 of FIG. 13 and the arousal estimation device 251 of FIG. 14, the reliability conversion unit 213 may be excluded. In that case, the reliability is used instead of the signal quality r; in the arousal estimation model learning unit 214 and arousal estimation unit 262.

In the above explanation, the instantaneous heart rate and its reliability estimated by this technology are used to estimate the arousal level, which is one of the emotional states of humans, i.e., concentration and relaxation states, which are the vertical axis directions described in Russell's circumplex model. This technique can also be applied to the estimation of pleasant and unpleasant states on the horizontal axis described by Russell's circumplex model, when the reliability of the input information can be defined.

### 4. Others

### <Outline in Related Art and Effects of Present Technology>

As described above, in the related art, even when the noise reduction process operates as expected, the peak position after the filtering processing may be shifted due to the poor S/N of the input signal. In this case, even in a case where the heart rate variability is analyzed using the time series data of the peak interval of the pulse wave signal, the analyzed heart rate variability greatly deviates from the result of the analysis of the heart rate variability by the electrocardiograph as a reference, and the accuracy of the healthcare application utilizing the heart rate variability has decreased.

PTL 1 proposes that a noise intensity is calculated on the basis of frequency spectrum analysis of a pulse wave signal, and an index is calculated on the basis of whether or not the quality of the pulse wave signal is equal to or higher than a reference value. However, in the technique described in PTL 1, since it may be impossible to evaluate the quality of each peak of the pulse wave signal, the detection accuracy of the heart rate by peak detection decreases.

In addition, PTL 2 proposes that a pulse wave signal is divided to acquire a plurality of sub signal segments, and it is determined whether it is noise from self-similarity in the pulse wave signal of the sub signal segment. However, for example, body motion in a strong periodic exercise such as jogging and running is periodically and strongly superimposed as body motion noise on the pulse wave signal. Therefore, self-similarity is increased, and the body motion is erroneously detected as a pulse wave signal due to heartbeat. In addition, even in a case where the self-similarity analysis is applied to the pulse wave signal after the noise reduction process, it is difficult to completely reduce the noise by the noise reduction process, and thus, the remaining periodic noise is a factor of erroneous determination of the self-similarity analysis.

In an aspect of the present technology, the first signal quality estimation is made of the input biometric signal, and the second signal quality estimation is made of the biometric signal after the noise reduction process. Then, the heart rate is estimated on the basis of the biometric signal after the noise reduction process, and the reliability of the heart rate is estimated on the basis of the result of the first signal quality estimation and the result of the second signal quality estimation.

Therefore, in an aspect of the present technology, by using the result of the first signal quality estimation and the result of the second signal quality estimation, it is determined whether or not the signal waveform for each heartbeat is derived from heartbeat. Therefore, it is possible to estimate the reliability of the heart rate (peak detection) at the detected peak position with high accuracy. That is, in the estimation of the heart rate from the pulse wave signal, it is possible to estimate the signal quality of the peak of the pulse wave signal for each heartbeat. With this arrangement, the heart rate can be estimated with high accuracy.

In other aspects of the technology, an estimation model for estimating human emotional states is learned using the heart rate and the reliability of the heart rate estimated by one aspect of the technology described above.

This enables highly accurate estimation of human emotional states.

### <Configuration Example of Computer>

The above-described series of processing can be executed by hardware or software. In a case where the series of processing is executed by software, a program constituting the software is installed from a program recording medium to a computer incorporated in dedicated hardware, a general-purpose personal computer, or the like.

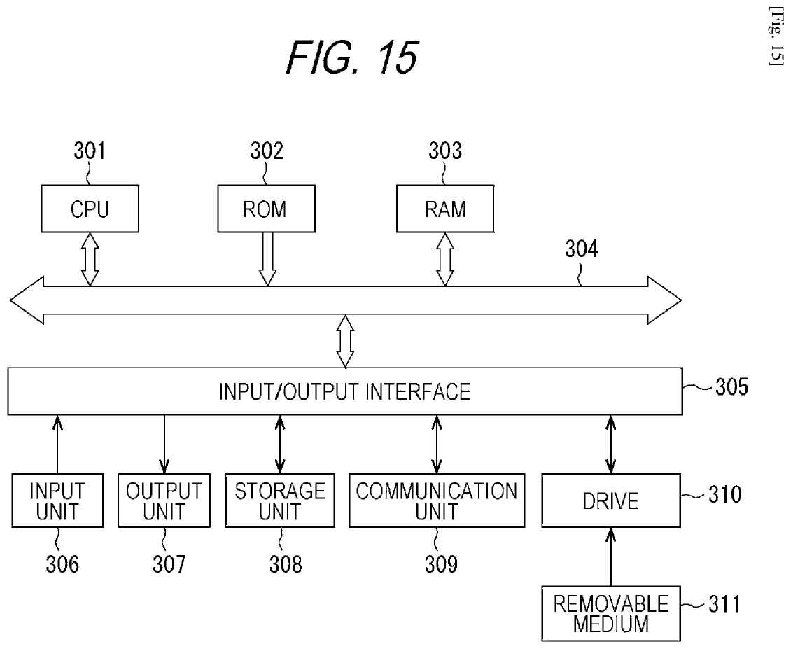

FIG. 15 is a block diagram illustrating a configuration example of hardware of a computer that executes the above-described series of processes by a program.

A central processing unit (CPU) 301, a read only memory (ROM) 302, and a random-access memory (RAM) 303 are mutually connected by a bus 304.

An input/output interface 305 is further connected to the bus 304. An input unit 306 including a microphone, a keyboard, a mouse, and the like, and an output unit 307 including a display, a speaker, and the like are connected to the input/output interface 305. Furthermore, a storage unit 308 including a hard disk, a nonvolatile memory, and the like, a communication unit 309 including a network interface and the like, and a drive 310 that drives a removable medium 311 are connected to the input/output interface 305.

In the computer configured as described above, for example, the CPU 301 loads a program stored in the storage unit 308 into the RAM 303 via the input/output interface 305 and the bus 304 and executes the program, whereby the above-described series of processing is performed.

The program executed by the CPU 301 is provided, for example, by being recorded in the removable medium 311 or via a wired or wireless transmission medium such as a local area network, the Internet, or digital broadcasting, and is installed in the storage unit 308.

Note that the program executed by the computer may be a program in which processing is performed in time series in the order described in the present specification, or may be a program in which processing is performed in parallel or at necessary timing such as when a call is made.

Note that, in the present specification, a system means a set of a plurality of components (devices, modules (parts), etc.), and it does not matter whether or not all the components are in the same housing. Therefore, a plurality of devices housed in separate housings and connected via a network and one device in which a plurality of modules is housed in one housing are both systems.

In addition, the effects described in the present specification are merely examples and are not limited, and other effects may be provided.

The embodiments of the present technology are not limited to the above-described embodiments, and various modifications can be made without departing from the gist of the present technology.

For example, the present technology can have a configuration of cloud computing in which one function is shared and processed in cooperation by a plurality of devices via a network.

In addition, each step described in the above-described flowchart can be executed by one device a can be shared and executed by a plurality of devices.

Furthermore, in a case where a plurality of processes is included in one step, the plurality of processes included in the one step can be executed by one device or can be shared and executed by a plurality of devices.

### <Combination Example of Configuration>

Note that the present technology can have the following configurations.

(1)

A signal processing device including:
a first signal quality estimation unit that makes a first signal quality estimation of an input biometric signal,
a second signal quality estimation unit that makes a second signal quality estimation of the biometric signal after a noise reduction process, and
a heart rate estimation unit that estimates a heart rate on based on the biometric signal after the noise reduction process and estimates a reliability of the estimated heart rate based on a result of the first signal quality estimation and a result of the second signal quality estimation.
   (2)

The signal processing device according to (1), in which
the heart rate estimation unit detects a peak position of the biometric signal and estimates the reliability of the estimated heart rate at the detected peak position.
   (3)

The signal processing device according to (1) or (2), in which the heart rate estimation unit estimates that the reliability of the estimated heart rate is high in a case where signal quality in the result of the first signal quality estimation and signal quality in the result of the second signal quality estimation are high.

(4)

The signal processing device according to (2) or (3), further including:
a periodicity analysis unit that performs a periodic analysis of the biometric signal after the noise reduction process,
the heart rate estimation unit estimates the reliability of the estimated heart rate based on the result of the first signal quality estimation, the result of the second signal quality estimation, and a result of a periodic analysis of the biometric signal after the noise reduction process.
   (5)

The signal processing device according to (4), further including:
a noise reduction processing unit that performs the noise reduction process on the biometric signal.
   (6)

The signal processing device according to (5), further including:
a body motion state analysis unit that analyzes, based on body motion information, about a user, acquired by a sensor, a body motion state of the user.
   (7)

The signal processing device according to (6), in which
in a case where it is analyzed that a body motion of the user is larger than a first threshold value, at least one of the noise reduction processing unit, the second signal quality estimation unit, or the periodicity analysis unit stops a process.
   (8)

The signal processing device according to (6), in which
in a case where it is analyzed that signal quality in the result of the first signal quality estimation is high and a body motion of the user is smaller than a second threshold value, at least one of the noise reduction processing unit, the second signal quality estimation unit, or the periodicity analysis unit stops a process.
   (9)

A signal processing device according to any one of (1) to (8), further comprising:
a correction processing unit that corrects the estimated heart rate based on the estimated reliability of the estimated heart rate.
   (10)

The signal processing device according to any one of (1) to (9), in which the biometric signal is a pulse wave signal.

(11)

The signal processing device according to any one of (1) to (10), in which the signal processing device is provided in a wearable housing.

(12)

A signal processing method executed by a signal processing device, the method including:
making first signal quality estimation of an input biometric signal, making second signal quality estimation of the biometric signal after a noise reduction process, and
estimating a heart rate based on the biometric signal after the noise reduction process, and
estimating a reliability of the estimated heart rate based on a result of the first signal quality estimation and a result of the second signal quality estimation.
   (13)

A program for causing a computer to function as:
a first signal quality estimation unit that makes first signal quality estimation of an input biometric signal,
a second signal quality estimation unit that makes second signal quality estimation of the biometric signal after a noise reduction process, and
a heart rate estimation unit that estimates a heart rate based on the biometric signal after the noise reduction process and estimates a reliability of the estimated heart rate based on a result of the first signal quality estimation and a result of the second signal quality estimation.
   (14)

A learning device includes:
a first signal quality estimation unit that makes a first signal quality estimation of an input biometric signal,
a second signal quality estimation unit that makes a second signal quality estimation of the biometric signal after a noise reduction process,
a heart rate estimation unit that estimates a heart rate based on the biometric signal after the noise reduction process and estimates a reliability of the estimated heart rate based on a result of the first signal quality estimation and a result of the second signal quality estimation,
a correction processing unit that corrects the estimated heart rate based on the estimated reliability of the estimated heart rate, and
an estimation model learning unit that learns an estimation model for estimating a person's emotional state using the estimated heart rate and the reliability of the estimated heart rate.
   (15)

A signal processing device comprising:
a first signal quality estimator that estimates a first signal quality of an input biometric signal,
a second signal quality estimator that estimates a second signal quality of output signal from a noise reduction processor, and
a heart rate estimator that estimates a heart rate based on the output signal from the noise reduction processor and estimates a reliability of the estimated heart rate based on a result of the estimated first signal quality estimation and a result of the estimated second signal quality.
   (16)

The signal processing device according to (15), in which
the heart rate estimator detects a peak position of the output signal from the noise reduction processor and estimates the reliability of the estimated heart rate at the detected peak position.
   (17)

The signal processing device according to (15) or (16), in which the heart rate estimation unit estimates that reliability of the heart rate is high in a case where signal quality in a result of the first signal quality estimation and signal quality in a result of the second signal quality estimation are high.

(18)

The signal processing device according to (16) or (17), further comprising:
a periodicity analysis unit that performs a periodicity analysis of the biometric signal after the noise reduction process, in which
the heart rate estimation unit estimates reliability of the heart rate on the basis of a result of the first signal quality estimation, a result of the second signal quality estimation, and a result of a periodicity analysis of the biometric signal after the noise reduction process.
   (19)

The signal processing device according to (18), further comprising:
a noise reduction processing unit that performs the noise reduction process on the biometric signal.
   (20)

The signal processing device according to (19), further comprising:
a body motion state analysis unit that analyzes, on the basis of body motion information, about a user, acquired by a sensor, a body motion state of the user.
   (21)

The signal processing device according to (20), in which
in a case where it is analyzed that a body motion of the user is larger than a first threshold value, at least one of the noise reduction processing unit, the second signal quality estimation unit, or the periodicity analysis unit stops a process.
   (22)

The signal processing device according to (20), in which
in a case where it is analyzed that signal quality in a result of the first signal quality estimation is high and a body motion of the user is smaller than a second threshold value, at least one of the noise reduction processing unit, the second signal quality estimation unit, or the periodicity analysis unit stops a process.
   (23)

A signal processing device according to any one of (15) to (22), further comprising a correction processing unit that corrects the estimated heart rate based on the estimated reliability of the heart rate.

(24)

The signal processing device according to any one of (15) to (23), in which the biometric signal is a pulse wave signal.

(25)

The signal processing device according to any one of (15) to (24), in which the signal processing device is provided in a wearable housing.

(26)

A signal processing method executed by a signal processing device, the method including:
making first signal quality estimation of an input biometric signal, making second signal quality estimation of the biometric signal after a noise reduction process, and
estimating a heart rate on the basis of the biometric signal after the noise reduction process, and estimating reliability of the heart rate on the basis of a result of the first signal quality estimation and a result of the second signal quality estimation.
   (27)

A program for causing a computer to function as
a first signal quality estimation unit that makes first signal quality estimation of an input biometric signal,
a second signal quality estimation unit that makes second signal quality estimation of the biometric signal after a noise reduction process, and
a heart rate estimation unit that estimates a heart rate on the basis of the biometric signal after the noise reduction process and estimates reliability of the heart rate on the basis of a result of the first signal quality estimation and a result of the second signal quality estimation.
   (28)

A learning device comprising:
a first signal quality estimation unit that makes first signal quality estimation of an input biometric signal,
a second signal quality estimation unit that makes second signal quality estimation of the biometric signal after a noise reduction process,
a heart rate estimation unit that estimates a heart rate on the basis of the biometric signal after the noise reduction process and estimates reliability of the heart rate on the basis of a result of the first signal quality estimation and a result of the second signal quality estimation,
a correction processing unit that corrects the heart rate on the basis of the estimated reliability of the heart rate, and
an estimation model learning unit that learns an estimation model for estimating a person's emotional state using the estimated heart rate and the reliability of the estimated heart rate.
   (29)

The signal processing method according to (26), further comprising:
determining body motion of a user, based on body motion information about the user, acquired by a sensor.
   (30)

The signal processing method according to (26) and (29),
wherein when the result of the estimated first signal quality is high and the result of the estimated second signal quality is high, the reliability of the estimated heart rate is high.
   (31)

The signal processing method according to (26) and (29) to (30), further comprising: periodically performing an analysis of the input biometric signal after reducing the noise of the input biometric signal,
wherein the reliability of the estimated heart rate is based on the result of the first signal quality estimation, the result of the second signal quality estimation, and a result of the periodical analysis of the input biometric signal after reducing the noise of the input biometric signal.
   (32)

The signal processing method according to (26) and (29) to (31),
wherein reducing the noise of the input biometric signal comprises a noise reduction processor that performs a noise reduction process on the input biometric signal.
   (33)

The signal processing method according to (26) and (29) to (32), further comprising: correcting the estimated heart rate based on the reliability of the estimated heart rate.

(34)

The signal processing method according to (26) and (29) to (33), wherein the input biometric signal includes a pulse wave signal.

It should be understood by those skilled in the art that various modifications, combinations, sub-combinations and alterations may occur depending on design requirements and other factors insofar as they are within the scope of the appended claims or the equivalents thereof.

### REFERENCE SIGNS LIST

11 Wearable heart rate meter
21 Signal quality estimation unit
22 Noise reduction processing unit
23 Signal quality estimation unit
24 Periodicity analysis unit
25 Heart rate estimation unit
26 Correction processing unit
27 Overall control unit
31 Analysis window setting unit
32 Time feature amount calculation unit
33 Frequency feature amount calculation unit
34 Signal quality estimation processing unit
35 Signal quality estimation model storage unit
51 Signal quality estimation model learning unit
61 Signal quality estimation model learning unit
62 Data set storage unit
71 Signal quality label generation unit
81 Noise reduction processing unit
82 Heart rate estimation unit
83 Arithmetic unit
84 Comparison determination unit
101 Wearable heart rate meter
111 Body motion context analysis unit
112 Signal quality estimation unit
113 Signal quality estimation unit
114 Overall control unit
201 Device for learning an arousal estimation model
211 Data set storage
212 Instantaneous heart rate estimator
213 Reliability convertor
214 Arousal estimation model learning unit
251 Arousal estimation device
261 Arousal estimation model storage
262 Arousal estimator

What is claimed is:

1. A signal processing device, comprising:
a first signal quality estimator that estimates a first signal quality of an input biometric signal;
a second signal quality estimator that estimates a second signal quality of an output signal from a noise reduction processor; and
a heart rate estimator that estimates a heart rate based on the output signal from the noise reduction processor and estimates a reliability of the estimated heart rate based on a result of the estimated first signal quality and a result of the estimated second signal quality.

2. The signal processing device according to claim 1,
wherein the heart rate estimator detects a peak position of the output signal from the noise reduction processor and estimates the reliability of the estimated heart rate at the detected peak position.

3. The signal processing device according to claim 1,
wherein the heart rate estimator estimates a high reliability of the estimated heart rate when the result of the estimated first signal quality is high and the result of the estimated second signal quality is high.

4. The signal processing device according to claim 2, further comprising:
a periodical analyzer that periodically performs an analysis of the output signal from the noise reduction processor,
wherein the heart rate estimator estimates the reliability of the estimated heart rate based on the result of estimating the first signal quality, the result of estimating the second signal quality, and a result of the periodical analysis of the output signal from the noise reduction processor.

5. The signal processing device according to claim 4,
wherein the noise reduction processor performs a noise reduction process on the input biometric signal to obtain the output signal.

6. The signal processing device according to claim 5, further comprising:
a body motion state analyzer that determines body motion of a user, based on body motion information about the user, acquired by a sensor.

7. The signal processing device according to claim 6,
wherein when the body motion of the user is greater than a first threshold value, at least one of the noise reduction processor, the second signal quality estimator, or the periodical analyzer stops a process.

8. The signal processing device according to claim 7,
wherein when the result of the estimated first signal quality is high and the body motion of the user is lower than a second threshold value, at least one of the noise reduction processor, the second signal quality estimator, or the periodical analyzer stops the process.

9. The signal processing device according to claim 1, further comprising:
a correction processor that corrects the estimated heart rate based on the reliability of the estimated heart rate.

10. The signal processing device according to claim 1,
wherein the input biometric signal includes a pulse wave signal.

11. The signal processing device according to claim 1,
wherein the signal processing device is provided in a wearable housing.

12. A signal processing method, the method comprising:
estimating a first signal quality of an input biometric signal;
reducing a noise of the input biometric signal;
estimating a second signal quality of the input biometric signal after reducing the noise of the input biometric signal; and
estimating a heart rate based on the input biometric signal after reducing the noise of the input biometric signal, and estimating a reliability of the estimated heart rate based on a result of estimating the first signal quality and a result of estimating the second signal quality.

13. A program executed by a processor that causes the processor to:
estimate a first signal quality of an input biometric signal;
estimate a second signal quality of the input biometric signal after a noise reduction process;
estimate a heart rate based on the input biometric signal after the noise reduction process; and
estimate a reliability of the estimated heart rate based on a result of estimating the first signal quality and a result of estimating the second signal quality.

14. A learning device comprising:
a first signal quality estimator that estimates a first signal quality of an input biometric signal;
a second signal quality estimator that estimates a second signal quality of an output signal from a noise reduction processor;
a heart rate estimator that estimates a heart rate based on the output signal from the noise reduction processor and estimates a reliability of the estimated heart rate based on a result of the first signal quality estimation and a result of the second signal quality estimation;
a correction processor that corrects the estimated heart rate based on the estimated reliability of the heart rate; and
a trained estimation model that estimates a person's emotional state using the estimated heart rate and the reliability of the estimated heart rate.

15. The signal processing method according to claim 12, further comprising:
determining body motion of a user, based on body motion information about the user, acquired by a sensor.

16. The signal processing method according to claim 12,
wherein when the result of the estimated first signal quality is high and the result of the estimated second signal quality is high, the reliability of the estimated heart rate is high.

17. The signal processing method according to claim 12, further comprising:
periodically performing an analysis of the input biometric signal after reducing the noise of the input biometric signal,
wherein the reliability of the estimated heart rate is based on the result of estimating the first signal quality, the result of estimating the second signal quality, and a result of the periodical analysis of the input biometric signal after reducing the noise of the input biometric signal.

18. The signal processing method according to claim 12,
wherein reducing the noise of the input biometric signal comprises a noise reduction processor that performs a noise reduction process on the input biometric signal.

19. The signal processing method according to claim 12, further comprising:
correcting the estimated heart rate based on the reliability of the estimated heart rate.

20. The signal processing method according to claim 12,
wherein the input biometric signal includes a pulse wave signal.
